(19) United States
(12) Patent Application Publication
(10) Pub. No.: US 2024/0382636 A1
(43) Pub. Date: Nov. 21, 2024

(54) ADAPTIVE FOOTWEAR SANITIZATION DEVICE

(71) Applicant: Aleksandar Milutin Serdar, Woodstock, GA (US)

(72) Inventor: Aleksandar Milutin Serdar, Woodstock, GA (US)

(21) Appl. No.: 18/319,807

(22) Filed: May 18, 2023

(51) Int. Cl. A61L 2/24; A61L 2/10; H05B 47/115; H05B 47/155

(52) U.S. Cl. CPC A61L 2/24; A61L 2/10; H05B 47/115; H05B 47/155; A61L 2202/11; A61L 2202/14

(57) ABSTRACT

An adaptive footwear sanitization device is disclosed. The device may include a base plate having a first portion and a second portion. The device may further include a plurality of first light sources disposed in the first portion and a plurality of second light sources disposed in the second portion. The device may further include a detection unit configured to detect a footwear presence on a base plate top surface. The device may additionally include a controller that may identify one or more first light sources from the plurality of first light sources covered by the footwear based on inputs obtained by the detection unit. The controller may activate the one or more first light sources and the plurality of second light sources responsive to identifying the one or more first light sources. The first and second light sources may emit light to sanitize footwear bottom portion.

### TECHNICAL FIELD

The present disclosure relates to a sanitization device, and more specifically to a footwear sanitization device that may adaptively emit ultraviolet light to a footwear based on footwear profile.

### BACKGROUND

People spend considerable effort and resources in maintaining hygienic and healthy lifestyle. For example, users wash or sanitize their hands when they visit public places like restaurants, gas stations, airports, etc. Regular hand sanitization may assist in eliminating bacteria that may accumulate on the hands. Exposure to bacteria may be more pronounced in public places as substantial number of people usually visits these places.

While users may regularly sanitize their hands, most users may not clean or sanitize their footwear. Lack of footwear sanitization may result in propagation of bacteria. For example, a user may accumulate bacteria on the footwear when the user visits a public place. If the user does not sanitize the footwear before entering home, the accumulated bacteria on the footwear may affect user health and/or health of user's family members.

Thus, there is a need for a footwear sanitization device that may be easily accessible to users, and may assist in eliminating bacteria that may accumulate on the footwear.

It is with respect to these and other considerations that the disclosure made herein is presented.

### BRIEF DESCRIPTION OF THE DRAWINGS

The detailed description is set forth with reference to the accompanying drawings. The use of the same reference numerals may indicate similar or identical items. Various embodiments may utilize elements and/or components other than those illustrated in the drawings, and some elements and/or components may not be present in various embodiments. Elements and/or components in the figures are not necessarily drawn to scale. Throughout this disclosure, depending on the context, singular and plural terminology may be used interchangeably.

FIG. 1 depicts an example environment in which techniques and structures for providing the systems and methods disclosed herein may be implemented.

FIG. 2 depicts a block diagram of an example adaptive footwear sanitization device in accordance with the present disclosure.

FIG. 3 depicts an example adaptive footwear sanitization device having a rotatable light source unit in accordance with the present disclosure.

FIG. 4 depicts an example adaptive footwear sanitization device having a plurality of shutters in accordance with the present disclosure.

FIG. 5 depicts an example adaptive footwear sanitization device having a plurality of example rotatable light source units in accordance with the present disclosure.

FIG. 6 depicts selective illumination of one or more rotatable light source units from the plurality of rotatable light source units of FIG. 5, in accordance with the present disclosure.

FIG. 7 depicts a flow diagram of an example method for sanitizing a footwear in accordance with the present disclosure.

FIG. 8 depicts another example adaptive footwear sanitization device in accordance with the present disclosure.

FIG. 9 depicts an isometric view of a base plate of the adaptive footwear sanitization device of FIG. 8 in accordance with the present disclosure.

FIG. 10 depicts an isometric view of a plurality of fixed shutters in accordance with the present disclosure.

FIG. 11 depicts an exploded view of the adaptive footwear sanitization device of FIG. 8 in accordance with the present disclosure.

### DETAILED DESCRIPTION

### Overview

The present disclosure is directed towards an adaptive footwear sanitization device that may enable a user to sanitize footwear. The user may stand on the device wearing the footwear and sanitize footwear bottom surface. The device may include a body having a body top surface and a body bottom surface. The body top surface may include guide areas on which the user may stand to sanitize the footwear bottom surface. The device may further include one or more rotatable light source units that may be disposed in a body interior portion, and may be configured to emit ultraviolet light through the guide areas when the user stands on the guide areas. The device may detect footwear profile and control light emission through the guide areas such that the emitted light stays within footwear contours and may not contact user skin.

In some aspects, the device may further include a plurality of shutters that may be disposed between the body top surface and the rotatable light source units. The shutters may be configured to block ultraviolet light emitted by the rotatable light source units from passing through the guide areas. The device may be configured to control shutter movement (i.e., fully or partially open the shutters) based on the footwear profile such that the light emitted from the rotatable light source units stays within the footwear contours.

Each rotatable light unit may be shaped as cylinder or a cuboid, and may include a first portion and a second portion that may be disposed opposite to the first portion. The first portion may include a light emitter that may emit ultraviolet light. In a device default position (i.e., when the device may not be in use), the first portion (i.e., the light emitter) may face the body bottom surface. The device may activate the light emitter and cause the rotatable light source unit to axially rotate relative to a rotatable light source unit longitudinal axis when the device detects that the user may be standing on the guide areas. The device may axially rotate the rotatable light source unit such that the light emitter may face the guide areas, thus enabling the light emitter to emit light through the guide areas. The device may further rotate “back” the rotatable light source unit and deactivate the light emitter when the light may be emitted on the footwear bottom surface for a predefined time duration (e.g., 4 to 8 seconds).

In some aspects, the device may not include the plurality of shutters to block the light emitted from the rotatable light source units, and instead the device may include a plurality of rotatable light source units that may be rotated independently of each other. In this case, the device may select one or more rotatable light source units from the plurality of rotatable light source units to activate and rotate, based on the footwear profile. The device may select the one or more rotatable light source units such that the light emitted from respective light emitters may stay within the footwear contours.

In further aspects, the device may be configured to detect if the user may be standing barefoot on the guide areas. Responsive to detecting that the user may be standing barefoot on the guide areas, the device may control intensity of light emitted from the rotatable light source units.

In additional aspects, the device may include a user interface that may display predetermined notifications. For example, the user interface may display a welcome note when the user stands on the body top surface. The user interface may further display instructions to use the device, error notification if the user may not be standing on the guide areas, etc. The device may further include a proximity sensor that may detect when the user may be in proximity to the device (e.g., within a predefined distance from the device). The device may cause the user interface to display the welcome note and/or instructions to the use the device when the proximity sensor detects that the user may be in proximity to the device.

In another embodiment of the present disclosure, the adaptive footwear sanitization device may include a base plate on which the user may stand wearing the footwear, or the user may place the footwear on the base plate. The base plate may include a plurality of first light sources and a plurality of second light sources. The plurality of first light sources and the plurality of second light sources may be disposed underneath a base plate top surface, and may be configured to emit Ultraviolet-C (UVC) light that may sanitize the footwear bottom surface when the footwear is placed over the base plate top surface.

In some aspects, the base plate top surface may include a plurality of fixed and slanted elongated shutters that may be disposed on a portion of the base plate top surface. A lateral axis of each shutter may be disposed at a predefined angle relative to the base plate top surface. The plurality of second light sources may be disposed underneath the plurality of fixed shutters such that the light emitted by the plurality of second light sources may fall on the footwear bottom surface at a predefined slant angle (and not at 90 degrees). In some aspects, the plurality of first light sources may be disposed under a first portion of the base plate top surface where a footwear heel may be placed, and the plurality of second light sources may be disposed under a second portion of the base plate top surface where a footwear outsole may be placed.

The device may further include a detection unit that may detect a footwear profile when the footwear may be placed on the base plate top portion. The device may activate one or more light sources from the plurality of first light sources to sanitize the footwear heal based on the footwear profile. In addition, the device may activate the plurality of second light sources when the footwear may be placed on the base plate top surface.

The device may further include a dome-shaped cover that may partially cover the base plate top surface. A cover interior surface may include another light source (e.g., a third light source) that may be activated when the footwear may be placed on the base plate top surface. The third light source too may emit UVC light and may be configured to sanitize footwear top portion when the footwear may be placed on the base plate top surface.

The present disclosure discloses an adaptive footwear sanitization device that may sanitize the footwear based on the footwear profile. In this manner, the device ensures that the ultraviolet light stays within the footwear contours and does not contact the user skin. Further, the device is lightweight and may be used conveniently at public places and/or user homes to sanitize footwear. Furthermore, the device outputs instructions to correctly use the device, so that the user may conveniently sanitize the footwear by using the device. These and other advantages of the present disclosure are provided in detail herein.

### Illustrative Embodiments

The disclosure will be described more fully hereinafter with reference to the accompanying drawings, in which example embodiments of the disclosure are shown, and not intended to be limiting.

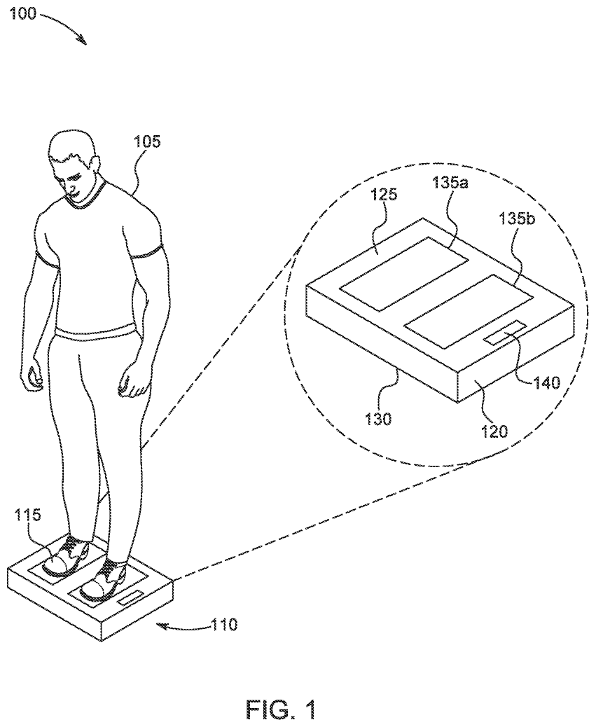

FIG. 1 depicts an example environment 100 in which techniques and structures for providing the systems and methods disclosed herein may be implemented. The environment 100 may include a user 105 who may be using an adaptive footwear sanitization device 110 (or device 110) to sanitize user footwear 115. Specifically, the user 105 may stand on the device 110 to sanitize a bottom surface of the footwear 115.

The footwear 115 may be, for example, shoes, boots, slippers, and/or the like. Further, the device 110 may located in a public place, for example, a restaurant, an airport, a grocery store, an office, a school, a hospital, and/or the like, or user home. The device 110 may be configured to sanitize footwear bottom surface (not shown) when the user 105 stands on the device 110 wearing the footwear 115. Specifically, the device 110 may emit ultraviolet light for a predetermined time duration (e.g., 3 to 8 seconds) towards the footwear bottom surface when the user 105 stands on the device 110. The ultraviolet light may sanitize or disinfect the footwear bottom surface, thus eliminating bacteria that may be accumulated on the footwear bottom surface. In some aspects, the ultraviolet light may be, for example, ultraviolet A (UVA), ultraviolet B (UVB), or ultraviolet C (UVC) light. In a preferred aspect, the device 110 may emit ultraviolet C (UVC) light.

The device 110 may include a body 120 having a body top surface 125 and a body bottom surface 130. The body 120 may be made of glass, plastic, fiber, aluminum, a combination thereof, and/or the like. In some aspects, one or more portions of the body 120 may be made of a first material, e.g., plastic or fiber, and remaining portions may be made of a second material, e.g., glass. The body 120 may be shaped as rectangle or square, with body edge length and/or width in a range of 12 to 24 inches. Further, a body thickness (i.e., a distance between the body top surface 125 and the body bottom surface 130) may be in a range of 0.5 to 1.5 inches. In some aspects, the body 120 may be hollow. In other aspects, one or more body portions may be hollow, and remaining body portions may be solid.

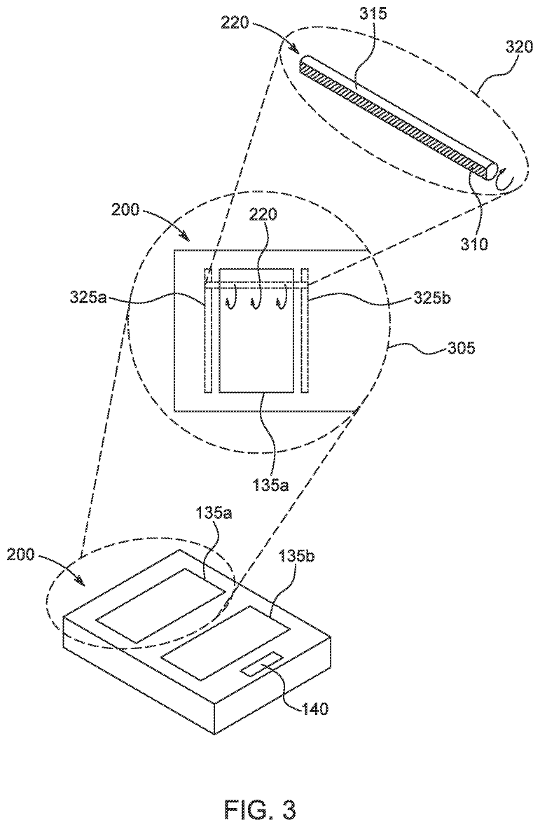

The body top surface 125 may include a first guide area 135a and a second guide area 135b (collectively referred to as guide areas 135). The guide areas 135 may be made of transparent or translucent glass, and may be shaped as a rectangle. Each guide area 135 may have a length in range of 10 to 12 inches, and a width in a range of 6 to 8 inches. In some aspects, the device 110 may emit ultraviolet light from the body top surface 125 via the guide areas 135. Specifically, the device 110 may include one or more light source units (shown as light source unit 220 in FIG. 3) disposed in a body interior portion underneath the guide areas 135 that may emit ultraviolet light towards the body top surface 125. The ultraviolet light emitted by the light source units may pass through the guide areas 135 and fall on the footwear bottom surface, thus sanitizing the footwear bottom surface. In this case, the user 105 may stand on the guide areas 135 to receive the ultraviolet light on the footwear bottom surface. In some aspects, the material of the guide areas 135 may allow the ultraviolet light to pass, and material of the remaining areas of the body top surface 125 may not allow the ultraviolet light to pass.

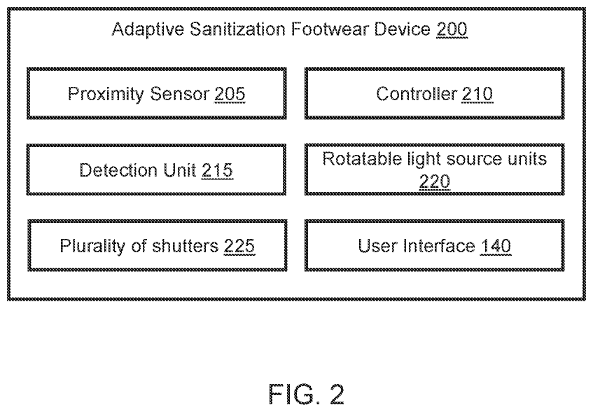

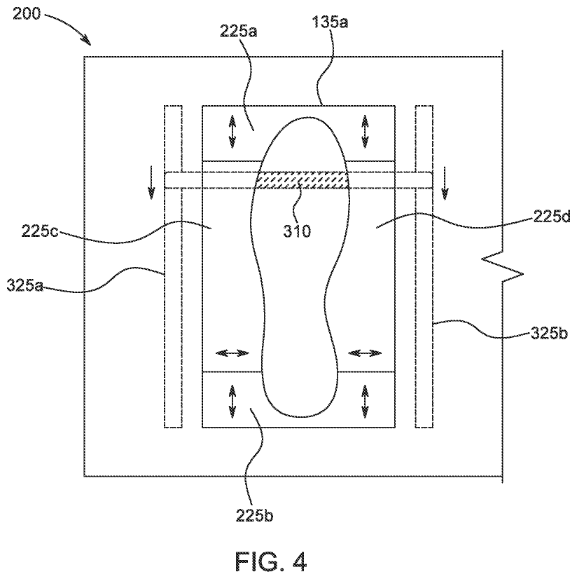

The device 110 may be configured to control exposure or emission of ultraviolet light through the guide areas 135 such that the emitted light may substantially or fully fall on the footwear bottom surface, and may not get emitted beyond footwear contours. Stated another way, the device 110 may be configured to control emission of ultraviolet light through the guide areas 135 based on footwear profile or shape such that the emitted ultraviolet light may stay within the footwear contours. In this manner, the device 110 may protect the user 105 (e.g., user's skin) from being exposed to the ultraviolet light. The device 110 may control ultraviolet light emission through the guide areas 135 by controlling the light source units and/or by using one or more shutters (shown as shutters 225 in FIG. 4) that may be disposed in the body interior portion. Detailed process of controlling ultraviolet light emission through the guide areas 135 is described in conjunction with FIGS. 2-6.

In some aspects, the body top surface 125 may further include a user interface 140 that may be configured to output notifications and/or receive user inputs. For example, the user interface 140 may output a welcome note when the user 105 stands on the guide areas 135 and/or when the user 105 may be in proximity to the device 110. The user interface 140 may further display additional information, e.g., date and/or time, instructions assisting the user 105 to correctly stand on the guide areas 135, error message if the user 105 stands on any other area different from the guide areas 135 on the body top surface 125, count of seconds remaining to sanitize the footwear bottom surface when the user 105 stands on the guide areas 135, error message when the device 110 may be broken or in a non-working condition, etc. The device 110 may additionally include a speaker (not shown) that may output audible notifications to assist the user 105 to use the device 110.

The device 110 may further include a proximity sensor (shown as proximity sensor 205 in FIG. 2) that may detect user presence when the user 105 may be in proximity to the device 110 (e.g., within a predefined distance from the device 110). Responsive to detecting the user presence, the device 110 may cause the user interface 140 to display one or more predetermined notifications. For example, the user interface 140 may display the welcome note and/or instructions to use the device 110 when the proximity sensor detects the user presence.

In some aspects, the device 110 may be placed flat on ground. In other aspects, the device 110 may be placed at an inclined angle relative to the ground. Stated another way, the device 110 may operate equally efficiently when the device 110 may be placed flat on the ground or inclined at an angle.

FIG. 2 depicts a block diagram of an example adaptive footwear sanitization device 200 (device 200) in accordance with the present disclosure. While describing FIG. 2, references may be made to FIGS. 3, 4, 5 and 6.

The device 200 may be same as the device 110. The device 200 may include a plurality of components including, but not limited to, a proximity sensor 205, a controller 210, a detection unit 215, one or more rotatable light source units 220, a plurality of shutters 225, and the user interface 140. The plurality of components may be communicatively and/or mechanically connected with each other.

The proximity sensor 205 may be disposed in the body interior portion and may be configured to detect user presence in proximity to the device 200, as described above in conjunction with FIG. 1. Responsive to detecting the user presence, the proximity sensor 205 may transmit a signal to the controller 210 indicating the user presence. The controller 210 may cause the user interface 140 to output/display one or more predetermined notifications (as described above), responsive to receiving the signal from the proximity sensor 205.

Although the description above (and as shown in FIG. 2) describes an aspect where the device 200 includes the proximity sensor 205, the present disclosure is not limited to the proximity sensor 205 being present in the device 200. Stated another way, the device 200 may work equally efficiently without the proximity sensor 205. In this case, the user interface 140 may display the predetermined notifications when the user 105 stands on the body top surface 125. For example, the user interface 140 may display the welcome note and/or the instructions to use the device 200 when the user 105 stands on the body top surface 125.

The detection unit 215 may be disposed in the body interior portion, and may be one or more of a camera (and an image processing unit), a capacitance measurement unit, a piezoelectric sensing unit, and/or the like. The detection unit 215 may be configured to detect that the user 105 may be standing on the body top surface 125. Responsive to detecting that the user 105 may be standing on the body top surface 125, the detection unit 215 may determine whether the user 105 may be standing on the guide areas 135. The detection unit 215 may transmit an error signal to the controller 210 when the detection unit 215 determines that the user 105 may not be standing on the guide areas 135. The controller 210 may cause the user interface 140 to display an error notification and/or instructions to correctly stand on the guide areas 135, responsive to receiving the error signal from the detection unit 215.

On the other hand, the detection unit 215 may further determine footwear profile when the detection unit 215 determines that the user 105 may be standing on the guide areas 135. Stated another way, the detection unit 215 may determine shape and dimensions of the footwear 115 when the user 105 may be standing on the guide areas 135. In some aspects, the detection unit 215 may determine the footwear profile by using images captured by the camera and executing edge detection algorithm(s) by using the image processing unit. In other aspects, the detection unit 215 may determine the footwear profile by measuring capacitance (and their difference thereof) at a plurality of points on the guide areas 135 by using the capacitance measurement unit and using the measured capacitance to map the footwear profile. In yet another aspect, the detection unit 215 may determine the footwear profile by measuring mechanical stresses at the plurality of points on the guide areas 135 by using the piezoelectric sensing unit and using the measured mechanical stresses to map the footwear profile.

Responsive to determining that the user 105 may be standing on the guide areas 135 and the footwear profile, the detection unit 215 may transmit an activation signal to the controller 210. The activation signal may include the determined footwear profile. Responsive to receiving the activation signal, the controller 210 may activate the rotatable light source units 220 such that ultraviolet light may be emitted through the guide areas 135 towards the footwear bottom surface. The concept of light emission through the guide areas 135, and structure of the rotatable light source units 220 may be understood in conjunction with FIGS. 3-6 described below.

FIG. 3 depicts the device 200 having the rotatable light source unit 220 disposed in the body interior portion underneath the guide areas 135, as shown in view 305. Although the view 305 depicts the rotatable light source unit 220 being disposed underneath the first guide area 135a, a similar rotatable light source unit (not shown) unit may be disposed underneath the second guide area 135b.

The rotatable light source unit 220 may be shaped as a cylinder or a cuboid, and may be made of plastic, aluminum, fiber, and/or the like. The rotatable light source unit 220 may be disposed in the body interior portion, directly underneath the first guide area 135a. In some aspects, the rotatable light source unit 220 may be disposed parallel to top and bottom edges of the first guide area 135a and may have a length equivalent to (or slightly more than) a first guide area width as shown in the view 305. Further, diameter/width of the rotatable light source unit 220 may depend on the thickness of the body 120.

The rotatable light source unit 220 may be configured rotate axially relative to a rotatable light source unit longitudinal axis. Further, in some aspects, the rotatable light source unit 220 may include a first portion 310 and a second portion 315, as shown in view 320. The second portion 315 may be disposed opposite to the first portion 310. The first portion 310 may include a light emitter that may be configured to emit ultraviolet light (e.g., ultraviolet-C light). Hereinafter, the first portion 310 is referred to as the light emitter 310.

In some aspects, in a default rotatable light source unit position, the light emitter 310 may face towards the body bottom surface 130. Stated another way, the light emitter 310 may be positioned away from the first guide area 135a in the default rotatable light source unit position (i.e., when the device 200 may not be in use). Responsive to receiving the activation signal from the detection unit 215, the controller 210 may activate (i.e., switch ON) the light emitter 310 and cause the rotatable light source unit 220 to axially rotate such that the light emitter 310 may face the first guide area 135a. In this manner, the light emitter 310 may emit ultraviolet light to the footwear bottom surface (that may be facing the guide areas 135 when the user 105 may be standing on the guide areas 135).

In some aspects, the device 200 may further include rails 325a, 325b (collectively referred to as rails 325) that may be disposed in the body interior portion underneath the first guide area 135a. The rails 325a, 325b may be disposed adjacent to left and right edges of the first guide area 135a, along a first guide area length. In some aspects, the rails 325a, 325b may be configured to enable the rotatable light source unit 220 to slide along the first guide area length when the light emitter 310 may be activated, such that the light emitter 310 may emit ultraviolet light along an entire footwear bottom surface area (and hence sanitize entire footwear bottom surface). Stated another way, the rotatable light source unit 220 may be configured to slide longitudinally along the footwear length by using the rails 325. In some aspects, responsive to receiving the activation signal from the detection unit 215, the controller 210 may cause the rotatable light source unit 220 to slide along the first guide area length (and hence the footwear length) by using the rails 325, in addition to activating the light emitter 310 and axially rotating the rotatable light source unit 220 as described above.

Although the description above (and as shown in FIG. 3) describes an aspect where the rails 325 may be disposed adjacent to the left and right edges of the first guide area 135a, the present disclosure is not limited to such rail arrangement. In another aspect (not shown), the rails 325 may be disposed adjacent to top and bottom edges of the first guide area 135a. In this case, the rotatable light source unit 220 may be disposed parallel to the left and right edges of the first guide area 135a, and may have a length equivalent to (or slightly more than) a first guide area length. Further, in this case, the rotatable light source unit 220 may be configured to slide laterally along a footwear width by using the rails 325 (when the controller 210 receives the activation signal from the detection unit 215). In yet another aspect (not shown), the device 200 may not include the rails 325, and instead the device 200 may include a plurality of rotatable light source units 220 that may cover an entire first guide area 135a. Since the plurality of rotatable light source units 220 may cover the entire first guide area 135a, the rotatable light source units 220 may not be required to slide along the footwear length and/or width to sanitize the footwear bottom surface. In this case, responsive to receiving the activation signal from the detection unit 215, the controller 210 may activate and rotate the plurality of rotatable light source units 220 simultaneously or sequentially to sanitize entire footwear bottom surface area.

In additional aspects, to ensure that the light emitted by the light emitter 310 stays within footwear contours, the controller 210 may control movement of the plurality of shutters 225 responsive to receiving the activation signal (that may include the footwear profile, as described above) from the detection unit 215. The plurality of shutters 225 are depicted in FIG. 4. Specifically, FIG. 4 depicts the device 200 having the plurality of shutters 225 (that is illustrated as shutters 225a, 225b, 225c and 225d).

The plurality of shutters 225 may be disposed in the body interior portion directly underneath the guide areas 135. In some aspects, the plurality of shutters 225 may be disposed between the body top surface 125 and the rotatable light source unit 220. The plurality of shutters 225 may be configured to block (completely or substantially) the light emitted by the light emitter 310 when the plurality of shutters 225 may be in closed position. The plurality of shutters 225 may be made of material such as plastic or aluminum, which may block the light emitted by the light emitter 310.

As shown in FIG. 4, the plurality of shutters 225 may include a first shutter 225a, a second shutter 225b, a third shutter 225c and a fourth shutter 225d. The first and second shutters 225a, 225b may be disposed on or in proximity to the top and bottom edges of the first guide area 135a and may be configured to slide along the first guide area length to block or unblock the first guide area 135a. Specifically, the first shutter 225a may move up towards first guide area top edge to be in an open position and unblock first guide area top portion. Similarly, the second shutter 225a may move down towards first guide area bottom edge to be in the open position and unblock first guide area bottom portion.

The third and fourth shutters 225c, 225d may be disposed on or in proximity to the left and right edges of the first guide area 135a and may be configured to slide along the first guide area width to block or unblock the first guide area 135a. Specifically, the third shutter 225c may move towards first guide area left edge to be in the open position and unblock first guide area left portion. Similarly, the fourth shutter 225d may move towards first guide area right edge to be in the open position and unblock first guide area right portion.

In some aspects, the plurality of shutters 225 may have curved edges (that may be located within the guide areas 135) that may be shaped as a standard footwear contour/edge. For example, as shown in FIG. 4, the first shutter 225a may have a curved edge that may be shaped as a footwear top edge, the second shutter 225b may have a curved edge that may be shaped as a footwear bottom edge, the third shutter 225c may have a curved edge that may be shaped as a footwear left edge, and the fourth shutter 225d may have a curved edge that may be shaped as a footwear right edge.

The controller 210 may be configured to control movement of the plurality of shutters 225 based on the footwear profile. Specifically, responsive to receiving the activation signal from the detection unit 215, the controller 210 may fully or partially open or close one or more shutters from the plurality of shutters 225 based on the footwear profile, such that the light emitted from the light emitter 310 may stay within the footwear contours. In this manner, the controller 210 may ensure that the light emitted by the light emitter 310 does not fall on user skin.

As shown in FIG. 4, by using the plurality of shutters 225, the light emitted from the light emitter 310 may pass through only those first guide area portions that may not be blocked by the plurality of shutters 225 (e.g., only those first guide area portions where the footwear bottom surface may be present). Further, as described above, the rotatable light source unit 220 (and hence the light emitter 310) may slide along the first guide area length to cover the entire footwear bottom surface and hence sanitize the footwear 115.

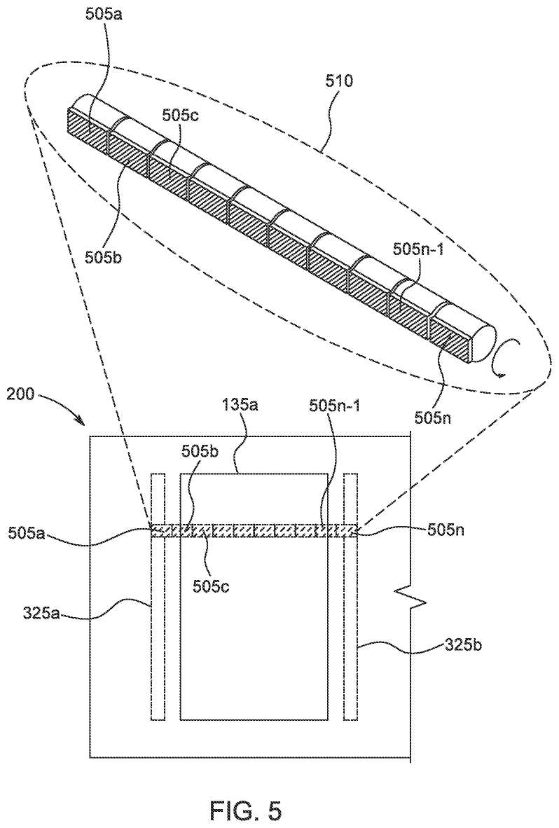

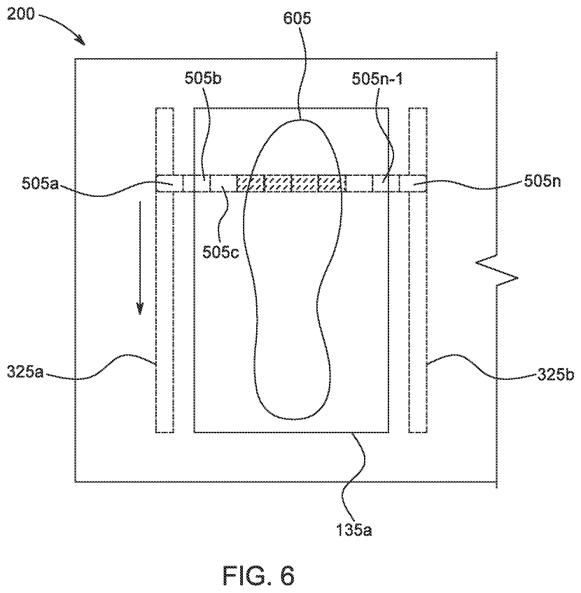

Although the description above describes an aspect where the device 200 may include the rotatable light source unit 220 and the plurality of shutters 225 may block the light emitted by the light emitter 310, the present disclosure is not limited to such arrangement. In some aspects, the device 200 may not include the rotatable light source unit 220 and may instead include an array of a plurality of static or stationary (i.e., non-rotatable) light emitters that may flood entire first guide area 135a with ultraviolet light. In this case, the plurality of shutters 225 may block light emitted by the plurality of static light emitters based on the footwear profile. In other aspects, the device 200 may not include the plurality of shutters 225 to block the light emitted by the light emitter 310 and may instead include a plurality of rotatable light source units that may be activated selectively based on the footwear profile, as shown in FIGS. 5 and 6.

FIG. 5 depicts a different embodiment of the device 200 that may include a plurality of rotatable light source units 505a-n. The plurality of rotatable light source units 505a-n may be disposed in the body interior portion and each rotatable light source unit 505a-n may be same as the rotatable light source unit 220. In some aspects, the rotatable light source units 505a and 505n may be attached to the rails 325, and remaining rotatable light source units may be disposed adjacent to each other along the first guide area width, as shown in view 510.

Each rotatable light source unit 505a-n may be configured to rotate independent of other rotatable light source units and may be controlled independently by the controller 210. Specifically, in this case, responsive to receiving the activation signal from the detection unit 215, the controller 210 may select one or more first rotatable light source units from the rotatable light source units 505a-n to activate and/or rotate based on the footwear profile, such that the light emitted from respective light emitters of the first rotatable light source units stays within the footwear contours, as shown in FIG. 6. Specifically, FIG. 6 depicts selective illumination of the first rotatable light source units. As shown in FIG. 6, the controller 210 may activate and/or rotate only those rotatable light source units that may “fall” within footwear contours 605 and may not activate and/or rotate remaining rotatable light source units, such that the emitted light stays within the footwear contours 605. The controller 210 may additionally cause the rotatable light source units 505a, 505n to slide along the first guide area length to enable the first rotatable light source units to emit light along the entire footwear bottom surface area.

In additional aspects, the controller 210 may activate/deactivate and/or rotate one or more additional (e.g., second) rotatable light source units from the plurality of rotatable light source units 505a-n as the rotatable light source units 505a, 505n slide along the first guide area length, to ensure that the entire footwear bottom surface is sanitized. For example, the controller 210 may activate the second rotatable light source units when the rotatable light source units 505a, 505n may be passing through a footwear contour top portion (which may have greater width), and may deactivate the second rotatable light source units when the rotatable light source units 505a, 505n may be passing through a footwear contour middle portion (which may have lesser width). In this manner, the controller 210 may ensure that the entire footwear bottom surface may be sanitized by the light emitted by the light emitters, and at the same time, the light may not escape the footwear contours 605.

In further aspects, the detection unit 215 may be configured to detect that the user 105 may be standing barefoot on the guide areas 135. The detection unit 215 may detect that the user 105 may be standing barefoot on the guide areas 135 by using the camera and the image processing unit described above. Responsive to detecting that the user 105 may be standing barefoot on the guide areas 135, the detection unit 215 may transmit an alert signal to the controller 210. Responsive to receiving the alert signal, the controller 210 may control (e.g., reduce) intensity of light emitted by the light emitter 310. In other aspects, responsive to receiving the alert signal, the controller 210 may not activate the light emitter 310 and/or rotate the rotatable light source unit 220. In this case, the device 200 may include one or more secondary rotatable light source units (not shown) of lower light intensity that may be activated and/or rotated by the controller 210 responsive to receiving the alert signal from the detection unit 215.

The device 200 may further include a power source (not shown) that may include rechargeable batteries. The power source may power different device components described above. The power source may be charged via utility power source and/or inductive wireless charging.

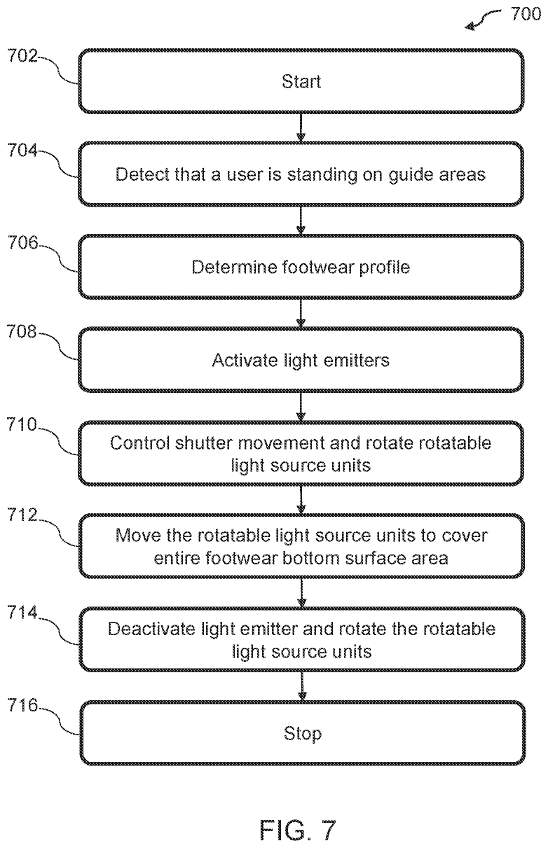

FIG. 7 depicts a flow diagram of an example method 700 for sanitizing the footwear 115 in accordance with the present disclosure. FIG. 7 may be described with continued reference to prior figures, including FIGS. 1-6. The following process is exemplary and not confined to the steps described hereafter. Moreover, alternative embodiments may include more or less steps that are shown or described herein and may include these steps in a different order than the order described in the following example embodiments.

Referring to FIG. 7, at step 702, the method 700 may commence. At step 704, the method 700 may include detecting, by the detection unit 215, that the user 105 may be standing on the guide areas 135. Responsive to detecting that the user 105 may be standing on the guide areas 135, the detection unit 215 may further determine the footwear profile at step 706. At step 708, the method 700 may include activating, by the controller 210, the light emitters 310. At step 710, the method 700 may include controlling, by the controller 210, movement of the plurality of shutters 225 and rotating “up” the rotatable light source units 220.

At step 712, the method 700 may include moving, by the controller 210, the rotatable light source units 220 along the entire first guide area length to cover and sanitize the entire footwear bottom surface area. At step 714, the method 700 may include deactivating, by the controller 210, the light emitters 310 and rotating back the rotatable light source units 220.

At step 716, the method 700 may stop.

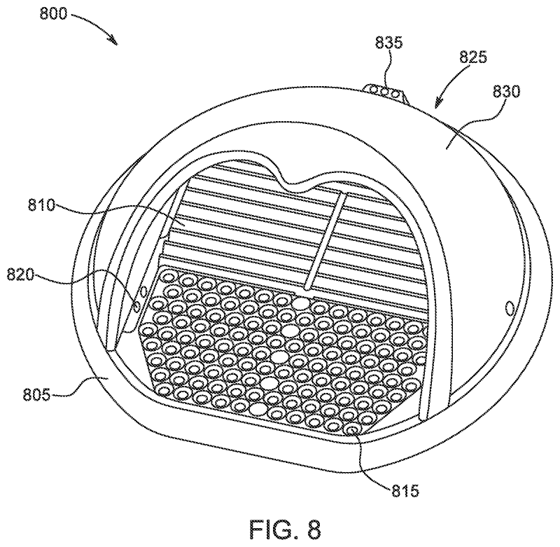

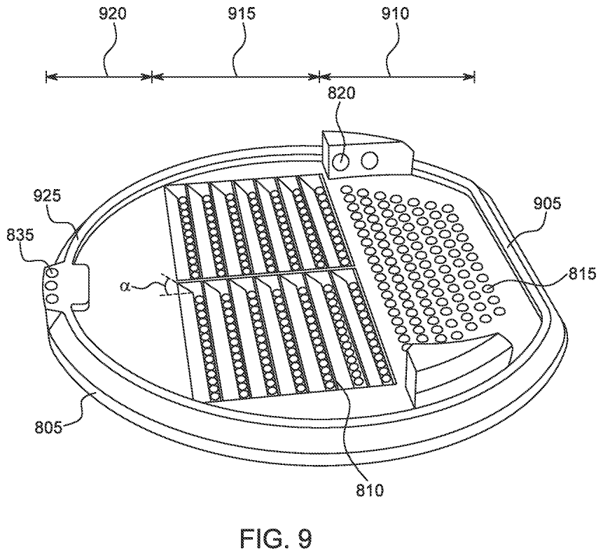

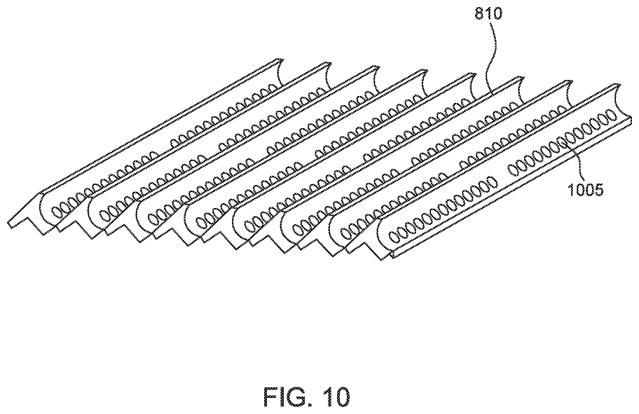

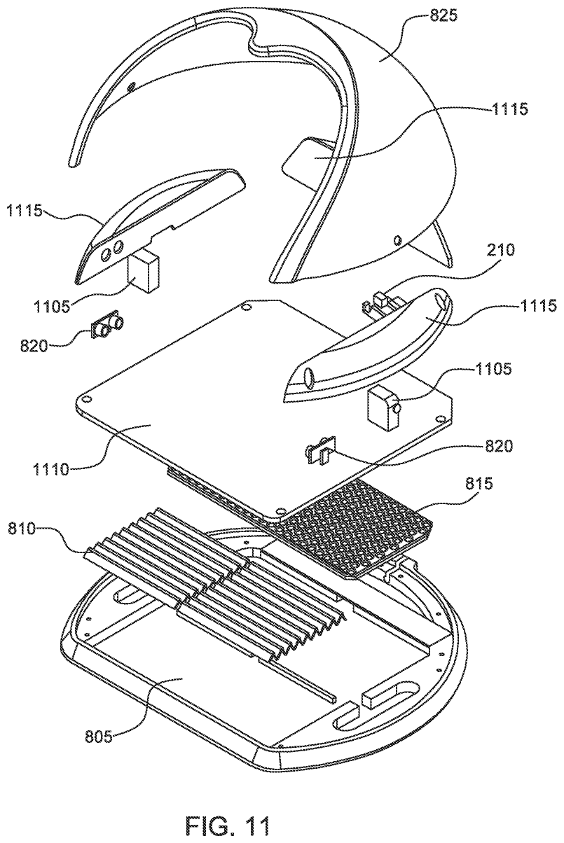

FIG. 8 depicts another example adaptive footwear sanitization device 800 (device 800) in accordance with the present disclosure. While describing FIG. 8, references may be made to FIGS. 9 and 10 that depict a base plate 805 and a plurality of elongated, slanted and fixed shutters 810 of the device 800. While describing FIG. 8, references may also be made to FIG. 11 that depicts an exploded view of the device 800.

The base plate 805 may be of any shape, e.g., rectangular, circular, oval-shaped, etc. In the exemplary aspect shown in FIGS. 8 and 9, the base plate 805 is shaped substantially circular with a flat or linear distal end 905. The base plate 805 may be of any diameter/width and/or thickness. Further, the base plate 805 may be made of any material including, but not limited to, plastic, metal, fiber, and/or the like.

The base plate 805 may include a first portion 910, a second portion 915 and a third portion 920 as shown in FIG. 9. The second portion 915 may be disposed between the first portion 910 and the third portion 920. The first portion 910 may have optimum dimensions (e.g., length and/or width) such that the user 105 may conveniently place footwear heel on the first portion 910. The second portion 915 may have optimum dimensions (e.g., length and/or width) such that the user 105 may conveniently place footwear outsole on the second portion 915.

The device 800 may further include a plurality of first light sources 815 that may be disposed in the first portion 910 underneath a base plate top surface. In some aspects, the base plate top surface may include a plate of glass or similar material, shown as a plate 1110 in FIG. 11, which may protect light sources that may be disposed underneath the base plate top surface. The first light sources 815 may be Light Emitting Diodes (LEDs) that may be configured to emit UVC light. The first light sources 815 may be disposed in a predefined pattern in the first portion 910, and each first light source may be equidistant from adjacent first light sources. In the exemplary aspect depicted in FIGS. 8, 9 and 11, the first light sources 815 may be disposed as a plurality of LED arrays. A count of first light sources in the plurality of first light sources 815 may depend on footwear sanitization device usage requirements.

The device 800 may further include a plurality of second light sources 1005 that may be disposed in the second portion 915. The plurality of second light sources 1005 is depicted in FIG. 10. The second portion 915 may include the plurality of elongated, slanted and fixed shutters 810 (or shutters 810) disposed on the base plate top surface, and the shutters 810 may include the second light sources 1005 installed underneath the base plate top surface. The second light sources 1005 may be similar to the first light sources 815, and may be configured to emit UVC light. The second light sources 1005 may be disposed in the second portion 915 and underneath the shutters 810 as multiple arrays of LEDs, as shown in FIG. 10.

In some aspects, each shutter of the plurality of shutters 810 may be slanted or disposed at a predefined angle “α” relative to the base plate top surface, as shown in FIG. 9. Due to the presence of “slanted” shutters, the light emitted by the second light sources 1005 may exit the base plate top surface at the angle “α”, and not “straight up” angled at 90 degrees relative to the base plate top surface.

The device 800 may further include one or more detection units 820 (or a detection unit 820). The detection unit 820 may be same as or different from the detection unit 215 described above. The detection unit 820 may be a camera, a proximity sensor, a capacitance measurement unit and a piezoelectric sensing unit. Although FIGS. 8, 9 and 11 depict the detection unit 820 being disposed at a base plate periphery, the present disclosure is not limited to the arrangement of the detection unit 820 depicted in FIGS. 8, 9 and 11. For example, the detection unit 820 may be disposed underneath the base plate top surface or one any other location on the device 800, without departing from the present disclosure scope.

The detection unit 820 may be configured to detect a footwear presence on the base plate top surface when the user 105 may stand on the base plate top surface wearing the footwear 115 or when the footwear 115 may be placed on the base plate top surface. The detection unit 820 may be further configured to determine a footwear profile (e.g., shape, size, etc.) when the footwear 115 may be placed on the base plate top surface. In additional aspects, the detection unit 820 may be configured to determine one or more first light sources from the plurality of first light sources 815 on which a footwear heel may be placed, based on the determined footwear profile. Specifically, the detection unit 820 may be configured to determine those first light sources (i.e., the one or more first light sources described above) that may be covered by the footwear heel or the footwear bottom portion when the footwear 115 may be placed on the base plate top surface. The detection unit 820 may be communicatively connected with a device controller (e.g., the controller 210) and may transmit inputs to the controller 210 when the detection unit 820 detects footwear presence on the base plate top surface.

In operation, the controller 210 may obtain the inputs from the detection unit 820 when the detection unit 820 detects footwear presence on the base plate top surface. The controller 210 may identify the one or more first light sources that may be covered by the footwear bottom portion based on the obtained inputs. Responsive to identifying the one or more first light sources, the controller 210 may activate the identified one or more first light sources. In addition, responsive to identifying the one or more first light sources or the footwear presence on the base plate top surface, the controller 210 may activate the plurality of second light sources 1005.

When the controller 210 activates the one or more first light sources, the one or more first light sources may emit UVC light and may sanitize the footwear heel part of the footwear bottom portion. Since the controller 210 may activate only those first light sources that may be underneath the footwear bottom portion and not remaining first light sources, probability of UVC light leaking out from footwear contours and reaching user's skin in greatly reduced.

In a similar manner, when the controller 210 activates the plurality of second light sources 1005, the second light sources 1005 may emit UVC light and may sanitize the footwear outsole part of the footwear bottom portion. Since the light emitted by the second light sources 1005 exits the base plate top surface through the slanted shutters 810 (that may be slanted in a “forward direction” towards the footwear outsole part), the UVC light emitted by the second light sources 1005 may not reach the user's skin and may only be confined to the footwear bottom portion.

In additional aspects, the device 800 may include a cover 825 that may be disposed over the base plate top surface, as shown in FIG. 8. The cover 825 may be made of carbon fiber, plastic, stainless steel, fiberglass, and/or the like. The cover 825 may be removably attached to the base plate periphery by one or more attachment means, shown as pin locks 1105 in FIG. 11. In an exemplary aspect, the cover 825 may be removably attached over the plate 1110 by using one or more side covers 1115 and the pin locks 1105, as shown in FIG. 11.

In some aspects, the cover 825 may be dome-shaped and may enclose or cover the third portion 920 fully and the second portion 915 fully or partially. The cover 825 may act as additional protection to ensure that light emitted from the first and/or second light sources 815, 1005 does not reach the user's skin. Cover front portion may be arc-shaped (having one arc, or two arcs with rubber lip as shown in FIG. 8) that may enable the user 105 to conveniently stand on the base plate top surface, without the cover front portion obstructing user legs.

The cover 825 may include an exterior cover surface 830 and an interior cover surface (not shown) disposed opposite to the exterior cover surface 830. The interior cover surface may face the base plate top surface. The cover 825 may further include another light source (e.g., a third light source, not shown) that may be disposed at the interior cover surface and may be configured to emit ultraviolet light towards the base plate top surface.

In some aspects, the controller 210 may activate the third light source responsive to identifying footwear presence on the base plate top portion and/or identifying the one or more first light sources described above. The third light source may emit ultraviolet light towards a footwear top portion, thus sanitizing the footwear top portion, when the controller 210 activates the third light source. In this manner, the device 800 may be configured to sanitize the footwear bottom portion and the footwear top portion simultaneously.

In further aspects, the device 800 may include one or more LEDs 835 configured to emit visible light in different colors, e.g., red, green and yellow. The LEDs 835 may be disposed at a base plate proximal end 925, as shown in FIG. 9. The controller 210 may activate the LEDs 835 based on different device operation states. For example, the controller 210 may activate a green LED when the user 105 may be standing on the base plate top surface and the device 800 may be sanitizing the footwear 115 or may be “ready” to sanitize. The controller 210 may activate a red LED when the device 800 may have sanitized the footwear 115 and may indicate to the user 105 to move away from the base plate top surface. The controller 210 may further activate a yellow LED when the device 800 may be faulty and/or may require maintenance.

In yet another aspect, the device 800 may include a mat (not shown) that may be attached to the base plate 805 or may be disposed in proximity to the base plate 805 (and not attached to the base plate 805). The user 105 may use the mat to remove excess debris from the footwear bottom portion.

In the above disclosure, reference has been made to the accompanying drawings, which form a part hereof, which illustrate specific implementations in which the present disclosure may be practiced. It is understood that other implementations may be utilized, and structural changes may be made without departing from the scope of the present disclosure. References in the specification to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a feature, structure, or characteristic is described in connection with an embodiment, one skilled in the art will recognize such feature, structure, or characteristic in connection with other embodiments whether or not explicitly described.

It should also be understood that the word “example” as used herein is intended to be non-exclusionary and non-limiting in nature. More particularly, the word “example” as used herein indicates one among several examples, and it should be understood that no undue emphasis or preference is being directed to the particular example being described.

With regard to the processes, systems, methods, heuristics, etc. described herein, it should be understood that, although the steps of such processes, etc. have been described as occurring according to a certain ordered sequence, such processes could be practiced with the described steps performed in an order other than the order described herein. It further should be understood that certain steps could be performed simultaneously, that other steps could be added, or that certain steps described herein could be omitted. In other words, the descriptions of processes herein are provided for the purpose of illustrating various embodiments and should in no way be construed so as to limit the claims.

Accordingly, it is to be understood that the above description is intended to be illustrative and not restrictive. Many embodiments and applications other than the examples provided would be apparent upon reading the above description. The scope should be determined, not with reference to the above description, but should instead be determined with reference to the appended claims, along with the full scope of equivalents to which such claims are entitled. It is anticipated and intended that future developments will occur in the technologies discussed herein, and that the disclosed systems and methods will be incorporated into such future embodiments. In sum, it should be understood that the application is capable of modification and variation.

All terms used in the claims are intended to be given their ordinary meanings as understood by those knowledgeable in the technologies described herein unless an explicit indication to the contrary is made herein. In particular, use of the singular articles such as “a,” “the,” “said,” etc., should be read to recite one or more of the indicated elements unless a claim recites an explicit limitation to the contrary. Conditional language, such as, among others, “can,” “could,” “might,” or “may,” unless specifically stated otherwise, or otherwise understood within the context as used, is generally intended to convey that certain embodiments could include, while other embodiments may not include, certain features, elements, and/or steps. Thus, such conditional language is not generally intended to imply that features, elements, and/or steps are in any way required for one or more embodiments.

That which is claimed is:

1. A sanitization device comprising:
a base plate having a first portion and a second portion;
a plurality of first light sources disposed in the first portion;
a plurality of second light sources disposed in the second portion;
a detection unit configured to detect a presence of a footwear on a base plate top surface; and
a controller communicatively connected with the detection unit, wherein the controller is configured to:
obtain inputs from the detection unit;
identify one or more first light sources from the plurality of first light sources covered by the footwear based on the inputs; and
activate the one or more first light sources and the plurality of second light sources responsive to identifying the one or more first light sources,
wherein the one or more first light sources and the plurality of second light sources emit light to sanitize a footwear bottom portion when respective light sources are activated.

2. The sanitization device of claim 1 further comprising a plurality of shutters disposed on the base plate top surface in the second portion, wherein a lateral axis of each shutter is disposed at a predefined angle relative to the base plate top surface.

3. The sanitization device of claim 2, wherein the plurality of second light sources is disposed underneath the plurality of shutters.

4. The sanitization device of claim 1, wherein the detection unit comprises at least one of a camera, a proximity sensor, a capacitance measurement unit and a piezoelectric sensing unit.

5. The sanitization device of claim 1, wherein the controller is further configured to:
determine a footwear profile based on the inputs; and
identify the one or more first lights sources based on the footwear profile.

6. The sanitization device of claim 1, wherein the plurality of first light sources and the plurality of second light sources are configured to emit ultraviolet light.

7. The sanitization device of claim 1, wherein the base plate further comprises a third portion, wherein the second portion is disposed between the first portion and the third portion.

8. The sanitization device of claim 7 further comprising a cover disposed over the base plate top surface, wherein the cover encloses the second portion and the third portion.

9. The sanitization device of claim 8, wherein the cover is dome-shaped.

10. The sanitization device of claim 8, wherein the cover comprises a cover exterior surface and a cover interior surface, and wherein the cover interior surface faces the base plate top surface.

11. The sanitization device of claim 10, wherein the cover further comprises a third light source disposed at the cover interior surface, and wherein the third light source is configured to emit ultraviolet light towards the base plate top surface.

12. The sanitization device of claim 11, wherein the controller is further configured to activate the third light source responsive to identifying the one or more first light sources.

13. A sanitization device comprising:
a base plate having a first portion and a second portion;
a plurality of first light sources disposed in the first portion;
a plurality of second light sources disposed in the second portion;
a plurality of shutters disposed on a base plate top surface in the second portion, wherein a lateral axis of each shutter is disposed at a predefined angle relative to the base plate top surface, and wherein the plurality of second light sources is disposed underneath the plurality of shutters;
a detection unit configured to detect a presence of a footwear on the base plate top surface; and
a controller communicatively connected with the detection unit, wherein the controller is configured to:
obtain inputs from the detection unit;
identify one or more first light sources from the plurality of first light sources covered by the footwear based on the inputs; and
activate the one or more first light sources and the plurality of second light sources responsive to identifying the one or more first light sources,
wherein the one or more first light sources and the plurality of second light sources emit light to sanitize a footwear bottom portion when respective light sources are activated.

14. The sanitization device of claim 13, wherein the base plate further comprises a third portion, wherein the second portion is disposed between the first portion and the third portion.

15. The sanitization device of claim 14 further comprising a cover disposed over the base plate top surface, wherein the cover encloses the second portion and the third portion.

16. The sanitization device of claim 15, wherein the cover comprises a cover exterior surface and a cover interior surface, wherein the cover interior surface faces the base plate top surface, and wherein the cover interior surface comprises a third light source.

17. The sanitization device of claim 16, wherein the controller is further configured to activate the third light source responsive to identifying the one or more first light sources.

18. The sanitization device of claim 13, wherein the detection unit comprises at least one of a camera, a proximity sensor, a capacitance measurement unit and a piezoelectric sensing unit.

19. The sanitization device of claim 13, wherein the plurality of first light sources and the plurality of second light sources are configured to emit ultraviolet light.

20. A sanitization device comprising:
a base plate having a first portion, a second portion and a third portion, wherein the second portion is disposed between the first portion and the third portion;
a plurality of first light sources disposed in the first portion;
a plurality of second light sources disposed in the second portion;
a cover disposed over a base plate top surface, wherein the cover encloses the second portion and the third portion;
a detection unit configured to detect a presence of a footwear on the base plate top surface; and
a controller communicatively connected with the detection unit, wherein the controller is configured to:
obtain inputs from the detection unit;
identify one or more first light sources from the plurality of first light sources covered by the footwear based on the inputs; and
activate the one or more first light sources and the plurality of second light sources responsive to identifying the one or more first light sources,
wherein the one or more first light sources and the plurality of second light sources emit light to sanitize a footwear bottom portion when respective light sources are activated.
